(19) United States
(12) Patent Application Publication
(10) Pub. No.: US 2023/0119239 A1
(43) Pub. Date: Apr. 20, 2023

(54) OROMUCOSAL FILM COMPOSITIONS

(71) Applicants: Josef Masek, Brno (CZ); Eliska Masková, Brno (CZ); Robert C H Chisholm, London (GB)

(72) Inventors: Josef Masek, Brno (CZ); Eliska Masková, Brno (CZ); Robert C H Chisholm, London (GB)

(73) Assignees: ReForm Therapeutics Ltd., London (GB); Veterinary Research Institute, Brno (CZ)

(21) Appl. No.: 17/934,198

(22) Filed: Sep. 21, 2022

Related U.S. Application Data
(63) Continuation-in-part of application No. PCT/IB2022/052411, filed on Mar. 17, 2022.
(60) Provisional application No. 63/161,976, filed on Mar. 16, 2021.

(51) Int. Cl. A61K 9/00; A61K 9/70; A61K 31/569; A61K 38/26; A61K 31/5517; A61K 31/5513; A61K 31/167; A61K 31/381; A61K 31/445; A61K 31/137; A61K 47/38; A61K 47/10

(52) U.S. Cl. CPC A61K 9/006; A61K 9/7007; A61K 31/569; A61K 38/26; A61K 31/5517; A61K 31/5513; A61K 31/167; A61K 31/381; A61K 31/445; A61K 31/137; A61K 47/38; A61K 47/10

(57) ABSTRACT

The invention provides an oromucosal film composition comprising:
a therapeutically effective amount of one or more active pharmacutical ingredients selected from the group consisting of fluticasone, liraglutide, glucagone, midazolam, diazepam, lidocaine, articaine, bupivacaine, prilocaine, mepivacaine, epinephrine and salts thereof;
a pharmaceutically acceptable polymer; and
an absorption enhancer selected from the group consisting of diethylene glycol monoethyl ether, eugenol, bile acids, caprylocaproyl polyoxyl-8 glycerides, disodium ethylenediaminetetraacetic acid, salt and mixtures thereof, wherein the composition comprises at least diethylene glycol monoethyl and eugenol as absorption enhancers when it comprises epinephrine alone or together with one or more of lidocaine, articaine, bupivacaine, prilocaine, mepivacaine as active pharmacutical ingredients.

The fim compositions of the invention can be used for providing anesthesia, or for the treatment of diabetes, epilepsy, tooth pain, mouth ulcers, cardiac arrest, asthma, bronchial asthma, bronchitis, emphysema, respiratory infections, anaphylactic shock, or allergic reactions in a subject in need thereof.

### CROSS REFERENCE TO RELATED PATENT APPLICATION

This application is a continuation in part of International Application No. PCT/IB2022/052411 filed Mar. 17, 2022, which claims priority to provisional U.S. Application No. 63/161,976 filed Mar. 16, 2021, the contents of which are herein incorporated by reference in their entirety.

### FIELD OF THE INVENTION

This invention relates to oromucosal film compositions that provide convenient quick alternative delivery of certain active pharmaceutical ingredients.

### BACKGROUND TO THE INVENTION

Mucosal drug delivery is an alternative method of systemic drug delivery that offers numerous benefits over parenteral and conventional oral administration. Oral mucosal surfaces have been widely explored for systemic delivery of drugs. Drugs that are absorbed via mucosal surfaces directly enter the systemic circulation and bypass the gastrointestinal tract including first-pass metabolism in the liver. Rapid onset of drug action is an important advantage of mucosal route of administration.

Transmucosal delivery has been employed for administration of therapeutic small molecules and biomacromolecules like peptides, proteins, nucleic acids, as well as antigens and allergens. Transmucosal drug delivery brings many advantages. Oral mucosae, especially its sublingual region, buccal region, and non-keratinized oral regions are attractive sites for non-invasive administration of drugs due to high permeability, a lack of enzymatic barriers, mild pH environment, easy access for self-administration, and opportunities to avoid first-pass metabolism.

Anatomy, physiology, and barrier functions of mucosal surfaces play a critical role in mucosal drug delivery. All aspects should be taken into consideration when designing a mucosal drug delivery system. The mucosa of oral cavity is divided into the buccal, sublingual, gingival, palatal and labial regions. The mucosa of each region is of specific anatomical and functional characteristics. Oral mucosa consists of three layers: a stratified squamous epithelium, composed of several cell layers, below which lies the basement membrane, and finally the connective tissue divided into the lamina propria and submucosa, which comprise numerous vascular capillaries. Drugs absorbed via the oromucosal route of administration are absorbed through these capillaries and gain access to the systemic circulation.

Three major types of epithelium located in different regions of the oral cavity differ in the degree of keratinization—namely masticatory, specialized, and lining mucosa. The masticatory epithelium is keratinized (100-200 μm thick) and covers the gingival region and the hard palate. The specialized epithelium is stratified, keratinized, and covers the dorsal surface of the tongue. The lining mucosa covers buccal and sublingual regions of the oral cavity. The epithelial layer of the buccal and sublingual mucosa is non-keratinized, with variation in thickness 500-600 μm for buccal, 100-200 μm for sublingual mucosa (Hua S. Advances in Nanoparticulate Drug Delivery Approaches for Sublingual and Buccal Administration. Front Pharmacol. 2019 Nov. 5; 10:1328. doi: 10.3389/fphar.2019.01328). The lining mucosa exhibits high permeability for different drugs, and thus is an interesting site for drug administration. The permeability of buccal mucosa is approximately 4-4,000 times greater than that of the skin, but less than that of the intestine.

The oral epithelium is covered by a 70-100 μm thick film of saliva, the secretion from salivary glands. The daily production of saliva secreted into the oral cavity is between 0.5 and 2 mL. Continuous production of saliva significantly impacts drug residence time after administration within the oral cavity, phenomenon known as saliva washout (Patel V F, Liu F, Brown M B. Advances in oral transmucosal drug delivery. J Control Release. 2011 Jul. 30; 153(2):106-16. doi: 10.1016/j.jconrel.2011.01.027; Hillery A M, Park K. Drug Delivery: Fundamentals and Applications. 2nd ed. Boca Raton, USA: CRC Press by Taylor & Francis Group, LLC; 2016, 632).

Mucus is the intercellular ground matrix secreted by the sublingual and salivary glands, which is bound to the apical cell surface and acts as a protective layer for the cells below. It is also a visco-elastic hydrogel consisting of the water insoluble glycoproteins, water, and small quantities of different proteins, enzymes, electrolytes and nucleic acids. The mucus layer carries a negative charge due to a high content of the sialic acid and forms a strongly cohesive gel structure that binds to the epithelial cells. The mucus layer varies in thickness from 40 to 300 μm and it plays a critical role in the function of different mucoadhesive drug delivery systems which work on the principle of mucoadhesion, and thus prolong the dosage form retention time at the site of administration.

The rate of drug absorption following oromucosal administration is influenced by the permeability of the buccal and sublingual mucosa, physical-chemical properties of the delivered drug and other factors, namely the presence and properties of mucus, saliva production, movement of the oral tissues during speaking, food and drink intake etc.

Drug permeability through the oral cavity mucosa represents a major limiting factor in transmucosal drug delivery. Mechanically stressed areas are keratinized and impermeable to water, which makes such areas unfavorable for drug delivery. On the other hand, more permeable non-keratinized buccal and sublingual epithelia make such regions of the oral cavity attractive sites for drug delivery and a great number of active ingredients are currently being explored for transmucosal drug delivery (Magek et al.: Nanofibers in Mucosal Drug and Vaccine Delivery. 2018, IntechOpen, https://doi.org/10.5772/intechopen.82279).

Various absorption enhancers were shown to be important excipient in the formulations intended for the delivery of drugs via oral mucosal surfaces (e.g. Nicolazzo et al. Buccal penetration enhancers—How do they really work?, J Control Release. 2005 Jun. 20; 105(1-2):1-15. doi: 10.1016/j.jconrel.2005.01.024., Sohi et al. Critical evaluation of absorption enhancers for oral mucosal drug delivery, Drug Dev Ind Pharm. 2010 March; 36(3):254-82. doi: 10.1080/03639040903117348). Various absorption enhancers were formulated in oral films containing a number of active ingredients including epinpehrine in U.S. Patent Application Publication Nos. 20170348251A1 and 20070293581A1.

### SUMMARY OF THE INVENTION

The present invention provides oromucosal film compositions, particularly for emergency treatments. The invention also provides manufacturing methods for preparation of the film compositions.

In one aspect, the present invent provides an oromucosal film composition comprising:
a therapeutically effective amount of one or more active pharmacutical ingredients selected from the group consisting of liraglutide, glucagone, midazolam, diazepam, fluticasone, lidocaine, articaine, bupivacaine, prilocaine, mepivacaine, epinephrine and salts thereof;
a pharmaceutically acceptable polymer; and
an absorption enhancer selected from the group consisting of diethylene glycol monoethyl ether, eugenol, bile acids, caprylocaproyl polyoxyl-8 glycerides, disodium ethylenediaminetetraacetic acid salt, and mixtures thereof,
   wherein the composition comprises at least diethylene glycol monoethyl ether and eugenol as an absorption enhancers when it comprises epinephrine alone or together with one or more of lidocaine, articaine, bupivacaine, prilocaine, mepivacaine as active pharmacutical ingredients.

In another aspect, the present invent provides an oromucosal film composition consisting essentially of:
a therapeutically effective amount of one or more active pharmacutical ingredients selected from the group consisting of liraglutide, glucagone, midazolam, diazepam, fluticasone, lidocaine, articaine, bupivacaine, prilocaine, mepivacaine, epinephrine and salts thereof;
a pharmaceutically acceptable polymer; and
an absorption enhancer selected from the group consisting of diethylene glycol monoethyl ether, eugenol, bile acids, caprylocaproyl polyoxyl-8 glycerides, disodium ethylenediaminetetraacetic acid salt, and mixtures thereof,
   wherein the composition comprises at least diethylene glycol monoethyl ether and eugenol as an absorption enhancers when it comprises epinephrine alone or together with one or more of lidocaine, articaine, bupivacaine, prilocaine, mepivacaine as active pharmacutical ingredients.

In yet another aspect, the present invent provides an oromucosal film composition comprising:
a therapeutically effective amount of one or more active pharmacutical ingredients selected from the group consisting of liraglutide, glucagone, midazolam, diazepam, fluticasone, lidocaine, articaine, bupivacaine, prilocaine, mepivacaine, epinephrine and salts thereof;
a pharmaceutically acceptable polymer; and
an absorption enhancer selected from the group consisting of diethylene glycol monoethyl ether, eugenol, bile acids, caprylocaproyl polyoxyl-8 glycerides, disodium ethylenediaminetetraacetic acid salt, and mixtures thereof,
   wherein the composition comprises at least diethylene glycol monoethyl ether and eugenol as an absorption enhancers when it comprises epinephrine alone or together with one or more of lidocaine, articaine, bupivacaine, prilocaine, mepivacaine as active pharmacutical ingredients, and
   wherein the composition excludes an anti-crystallization agent, such as various sugar alcohols and di-alcohols, including, for example one or more of sorbitol, mannitol, xylitol, isomalt, and the like.

In some embodiments, the composition is a bi-layered or tri-layered film further comprising a mucoadhesive layer and/or a backing layer. In a bi-layered film, the first layer comprises one or more active pharmaceutical ingredients dispersed or dissolved in and/or disposed on a polymeric mucoadhesive layer, and a second layer which acts as a backing layer. Either the mucoadhesive layer or the polymeric layer comprising the active ingredient is the middle layer in case of a tri-layered composition. The film is intended for application to oral mucosa with an orientation that keeps the active ingredient-containing layer in direct, close contact with the oral mucosa surface.

In some embodiments, the composition further comprises one or more pharmaceutically acceptable excipients selected from the group consisting of pH modifiers, disintegrants, plasticizers, saliva stimulating agent, taste masking agents, flavoring agents, sweeting agent, coloring agent, antioxidants, stabilizers, anti-foaming and/or defoaming components and mixtures thereof. In some embodiments, one or more of the excipients are exclusively or partly present in the polymeric layer in case of a multi-layered composition. In other embodiments, one or more of the excipients are exclusively or partly present in the mucoadhesive layer of a multi-layered composition.

In some other embodiments, the composition comprises epinephrine as solid particles having a particle size in the range of from about 0.01 um to about 100 um.

In another aspect, the invention provides a method for preparation of an oromucosal film composition comprising:
providing a suspension or solution of one or more active pharmaceutical ingredients in a solvent/dispersant;
simultaneously or separately providing a suspension or solution of the polymer in a solvent/dispersant;
combining the active ingredient suspension/sloution and the polymer suspension/solution if prepared separately; and
removing the solvent/dispersant to obtain the polymeric layer.

In yet another aspect, the oromucosal film compositions of the invention may be prepared by preparation methods comprising solvent casting, hot melt extrusion, electro spinning, 3-dimensional or flexographic printing, spraying, combination of solvent casting and spraying, electrospraying, blow spinning, electroblowing, centrifugal spinning, or a combination of electrospinning and electrospraying.

In another aspect, a method of treating diabetes in a subject in need thereof is provided comprising administering to the subject a composition of the invention wherein one or more of liraglutide, glucagon and salts thereof are selected as the active pharmacutical ingredients.

In another aspect, a method of treating epilepsy in a subject in need thereof is provided comprising administering to the subject a composition of the invention wherein one or more of diazepam, midazolam and salts thereof are selected as the active pharmacutical ingredients.

In another aspect, a method of providing local anesthesia to a subject in need thereof is provided comprising administering to the subject a composition of the invention wherein one or more of lidocaine, articaine, bupivacaine, prilocaine, mepivacaine, and salts thereof are selected as the active pharmacutical ingredients.

In another aspect, a method of treating and/or preventing tooth pain or mouth ulcers in a subject in need thereof is provided comprising administering to the subject a composition of the invention wherein one or more of lidocaine, articaine, bupivacaine, prilocaine, mepivacaine, and salts thereof as the active pharmacutical ingredients. In some embodiments, the composition further comprises fluticasone.

In some embodiments, the composition comprising one or more of lidocaine, articaine, bupivacaine, prilocaine, mepivacaine, fluticasone and salts thereof as the active pharmacutical ingredient further comprises a low dose of epinephrine or a salt thereof.

In another aspect, a method of treating anaphylactic shock or cardiac arrest in a subject in need thereof is provided comprising administering to the subject a composition according to the invention wherein epinephrine or salts thereof are selected as the active pharmacutical ingredients.

### BRIEF DESCRIPTION OF THE DRAWINGS

FIG. 1 shows the design of a bi-layered film of the invention.

FIG. 2 shows the effect of permeation enhancers on permeation of Lidocaine ex vivo in porcine sublingual mucosa.

FIG. 3 shows pharmacokinetic profile of Lidocaine following the administration of a mucoadhesive film demonstrating the synergic effect of Transcutol® HP and Eugenol as permeation enhancers.

FIG. 4 shows permeation of Liraglutide through porcine sublingual mucosa ex vivo.

### DETAILED DESCRIPTION OF THE INVENTION

The absorption enhancers of the present invention that facilitate convenient quick alternative delivery of the active pharmaceutical ingredients in the form of oromucosal films include diethylene glycol monoethyl ether (transcutol®), eugenol, bile acids, caprylocaproyl polyoxyl-8 glycerides (labrosol®), disodium ethylenediaminetetraacetic acid, salts and mixtures thereof. Bile acids include deoxycholate sodium, taurocholate sodium, and glycocholate sodium.

The active pharmaceutical ingredients (API) used in the invention may be in the form of free base, salts, or mixtures thereof. These active ingedients are well known in the art and may be prepared by convention methods or obtained commercially.

The term “consisting essentially of” means that the composition of the claim is limited to the components specified in the claim and those additional components that do not materially affect the basic and novel characteristic(s) of the claimed composition.

The amount of the active ingredient used in the film depends on the intended use. The dosage levels of these active ingreients are well known in the art. For example, the amount of fluticasone included in the film compositions of the invention may be from 0.010 mg to 500 mg; the amount of liraglutide may be from 0.6 mg to 18 mg; the amount of glucagone may be from 0.5 mg to 15 mg; the amount of midazolam may be from 1.5 to 50 mg; the amount of diazepam may be from 2 mg to 50 mg; the amount of epinephrine may be from 1 mg to 20 mg; and the amount of local anesthetics lidocaine, articaine, bupivacaine, prilocaine, or mepivacaine may be from 0.5 mg to 60 mg.

Due to the vasoconstrictive properties of epinephrine a low dose of epinephrine may be used in combination with local anesthetics and/or fluticasone to prolonge the effect of anesthesia. A low dose of epinephrine may be from 0.005 mg to 1 mg.

The active pharmaceutical ingredients are dissolved/dispersed in and/or disposed on a polymeric layer which is a consistent homogeneous film or porous layer comprising of one or more polymers. The active pharmaceutical ingredient may be distributed uniformly throughout the polymeric layer in the form of solid dispersion or solutionor, disposed or embedded on the entire surface of the polymeric layer or only a portion of its surface.

In some embodiments, the active ingredients are in the form of particles which are embedded within the polymeric layer, thus forming a composite material of polymers and particles. In some other embodiments, the active ingredient particles are attached to, or disposed on the polymeric layer. The distribution of active ingredient particles may or may not be homogeneous.

In some embodiments, the polymeric layer is a porous layer comprising polymeric nanofibers and/or microfibers. When nano or microfibers are used, the active ingredient particles are deposited on the surface of fibers, and/or deposited into a mesh of polymeric nano or microfibers. The composite fiber-based polymeric film can be single, bi- or tri-layered, where there is a backing layer, composite active ingredient particle-containing layer (with or without mucoadhesive properties) and a layer of mucoadhesive micro or nanofibers. The backing layer can be the same size as the other layers or larger to form a mucoadhesive seal around the other layers.

In some embodiments the active pharmaceutical ingredient is in the form of solid particles which are at least partly included or embedded within the polymeric microfiber and/or nanofiber. In some other embodiments, the solid particles are dispersed in the polymeric layer but are not part of or included within the polymeric microfibers and/or nanofibers.

The active ingredient-containing polymer layer may be designed to dissolve in about 1 to about 30 minutes in the mouth, including more than 1 minute, more than 5 minutes, more than 10 minutes, more than 20 minutes or less than 30 minutes. In some embodiments, such as when used for local anesthesia, such as for dental anesthesia or mouth ulcers, or for diabetes or epilepsy the polymer layer may dissolve in more than 30 minutes and last up to 12 hours in the mouth.

For fast dissolving films low molecular weight hydrophilic polymers, such as polymers having a molecular weight between about 1,000 to 9,000 daltons, or polymers having a molecular weight up to 900,000 daltons may be used, optionally in combination with other suitable polymers. For slower dissolving films higher molecular weight polymers, such as those having a molecular weight in millions of daltons may be used, optionally in combination with other suitable polymers. Desired results can be achieved by adjusting the concentration of two or more types of polymers. Concentration of the polymers and the type/nature/solubility of the polymers used in each layer of the composition may be varied to control the amount of time the film composition resides on the mucosa. Disintegrants and plasticizers may be used to adjust erodibility of the film composition. Water soluble pharmaceutically acceptable excipients may be used to achieve desired erodibility/solubility of the film, particularly when insoluble or low solubility polymers are included.

In certain embodiments, absorption enhancers are formulated into the active ingredient-containing polymer layer. In certain other embodiments, absorption enhancers are formulated into the mucoadhesive or backing layer.

In certain embodiments, pH modifiers are formulated into the active ingredient-containing polymer layer. In certain other embodiments, pH modifiers are formulated into the mucoadhesive and/or backing layer.

In certain embodiments, absorption enhancers and the active pharmaceutical ingredients are mixed with the polymer to formulate a polymeric matrix so as to form a composite pharmaceutical film formulation. In certain other embodiments, absorption enhancers, pH modifying agents are formulated into a polymeric matrix comprising nano/microfibers and active ingredient particles are deposited in the polymeric matrix. In certain embodiments, absorption enhancers and pH modifying agents are formulated into a polymeric matrix and active ingredient particles are deposited or deposed on the polymeric matrix.

In certain embodiments, when polymeric nano/microfibers are used, mucoadhesive polymers are used in forming the fibers.

The weight ratio of the total active pharmacutical ingredients to the polymer(s) used in the polymeric layer may be from about 5% to about 50%, from about 10% to about 40%, from about 15% to about 35%, from about 15% to about 30%, or from about 15% to about 25%.

The active pharmaceutical ingredients dispersed in and/or disposed on the polymeric layer may have a particle size in the range of from about 0.01 um to about 100 um. In some embodiments the particle size may be from about 0.1 um to about 50 um, from about 1 um to about 40 um, from about 8 um to about 100 um, from about 10 um to about 100 um, from about 20 um to about 80 um, from about 20 um to about 30 um, or from about 0.1 um to 3 um.

In some embodiments, the polymers used in the polymeric layer may be rapidly or moderately dissolvable in the saliva. In some embodiments, the polymers may be water soluble or swellable. Suitable polymers include water-soluble synthetic polymers and co-polymers, polosynthetic polymers and co-polymers, and natural polymers. Examples of suitable polymers include cellulose derivatives, polyacrylates, poloxamers, polyethylene oxides, polyvinyl alcohols, povidone, poly-amino acids, gums and other natural polymers.

Polymers that may be used in the polymeric layer include hypromellose, hydroxypropyl cellulose, hydroxyethyl cellulose, carboxymethyl cellulose, carboxymethyl cellulose sodium, croscarmellose sodium, carbomer, polycarbophil, povidone, crospovidone, polyvinyl alcohol, polyethylene oxide, polyethylene glycol, sodium alginate, calcium alginate, xanthan gum, pectin, hyaluronic acid, sodium hyaluronate, tragacanth, guar gum, acacia gum, arabic gum, lectin, starch, gelatin, pullulan, carrageenan, chitosan, amino methacrylate copolymers, poloxamer, collagen, and mixtures thereof.

In case of a single layered or bi-layered film composition comprising a backing layer, the polymers used in the polymeric layer include one or more mucoadhesive polymers. A suitable mucoadhesive polymer is also used in the mucoadhesive layer of a bi-layered or tri-layered film.

Mucoadhesive polymers in the polymeric or mucoadhesive layer are used to enhanced retention of the film composition on the oral mucosa. Such polymers include soluble and insoluble, non-biodegradable and biodegradable polymers. These polymers can be hydrogels or thermoplastics, homopolymers, copolymers or blends, natural or synthetic. Examples of suitable polymers include the same polymers as listed above for polymeric layer provided they have mucoadhesive properties.

A backing layer is an occlusive layer designed to drive unidirectional release and absorption of epinephrine into the mucosa thereby decreasing amount of active ingredient required. A backing layer can also improve mechanical properties of the film to facilitate handling during production and use.

Backing layer may be formed using a hydrophobic, hydrophilic, erodible, low solubility or insoluble polymer, such as ethyl cellulose, hydroxyethyl cellulose, hydroxypropyl cellulose, methylcellulose, hypromellose, hypromellose phthalate, cellulose acetate, cellulose acetate phthalate, cellulose acetate butyrate, polyvinyl alcohol, povidone, crospovidone, polyethylene glycol, polyethylene oxide, polyvinyl alcohol-polyethylene glycol graft-copolymers, amino methacrylate copolymers, ethylene oxide, propylene oxide co-polymers, poly(vinyl acetate), poly(ethylene-co-vinyl) acetate, polycaprolactone, pharmaceutical wax, and mixtures thereof.

The mucoadhesive layer and/or backing layers may fully overlap with the polymeric layer or be larger than the polymeric layer such that they extend beyond and encompass the polymeric layer on all sides to facilitate effective mucoadhesion and/or prevent peripheral loss of the active pharmaceutical ingredients.

In some embodiments, the polymeric layer comprising active pharmacutical ingredients is printed onto the mucoadhesive and/or backing layer.

In some embodiments, mixtures of diethylene glycol monoethyl ether and eugenol are used as absorption enhancers to synergistically increase the bioavailability of active pharmacutical ingredients.

The thickness of the film composition may vary, depending on the number of layers and their thickness. The thickness and number of layers may be adjusted in order to vary the release/absorption of the active pharmacutical ingredients and erosion/residence of the film in the mouth. For example, the thickness may range from about 0.05 μm to about 2 mm, from about 0.1 to about 0.5 mm, from about 0.01 mm to about 1 mm, or greater than 0.1 mm, greater than 0.2 mm, or less than 0.5 mm, less than 0.2 mm, less than 0.1 mm, or less than 1 um. The thickness of each layer may vary from 0.1 to 99.9% of the overall thickness, or 1 to 99% of the overall thickness, or 10 to 90% of the overall thickness of the multi-layered composition. For example, the thickness of each layer may vary from 0.01 mm to 0.9 mm.

The amount of active pharmaceutical ingredient used in the active ingredient-containing layer may be from about 10% to about 30%, from about 12% to about 25%, from about 15% to about 20%, from about 15% to about 30%, or from about 15% to about 25%.

The amount of polymer used in the polymeric layer may be from about 5% to about 90%, from about 10% to about 85%, from about 25% to about 85%, from about 50% to about 75%, from about 55% to about 75%, or from about 60% to about 70%.

The amount of absorption enhancer used in the polymeric layer may be from about 1% to about 3%, from about 1% to about 20%, from about 2% to about 15%, from about 6% to about 12%, or from about 3% to about 10%.

The amount of antioxidant used in the polymeric layer may be from about 0.001% to about 10%, from about 0.005% to about 5%, from about 0.01% to about 3.5%, or from about 0.1% to about 3%. The amount of plasticizer used in the polymeric layer may be from about 2% to about 20%, from about 3% to about 18%, from about 4% to about 17%, from about 5% to about 20%, or from about 12% to about 18%.The amount of pH modifier used in the polymeric layer may be from about 1% to about 25%, from about 2% to about 20%, from about 3% to about 18%, from about 4% to about 17%, or from about 5% to about 15%.

Pharmaceutically Acceptable Excipients

The film composition may further comprise one or more pharmaceutically acceptable excipients, such as pH modifiers, binders, disintegrants, plasticizers, saliva stimulating agent, taste masking agents, flavoring agents, sweeting agent, coloring agent, antioxidants, stabilizers, anti-foaming and/or defoaming components and mixtures thereof. In some embodiments, the excipients are exclusively or partly present in the mucoadhesive layer and/or backing layer in case of a multilayered film. These excipients are well known in the art, such as those described in US20170348251A1.

The excipients may constitute up to about 80%, such as about 0.005% to 50%, or from about 1% to about 30% by weight of the composition.

Flavoring agents and/or sweeteners include acacia syrup, acesulfame K, alitame, anise, apple, aspartame, banana, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, fructose, fruit punch, ginger, glycyrrhetinate, glycyrrhiza syrup, grape, grapefruit, honey, isomalt, lemon, lime, lemon cream, monoammonium glyrrhizinate, maltol, mannitol, maple, marshmallow, menthol, mint cream, mixed berry, neohesperidine DC, neotame, orange, pear, peach, peppermint, peppermint cream, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, swiss cream, tagatose, tangerine, thaumatin, tutti fruitti, vanilla, walnut, watermelon, wild cherry, wintergreen, xylitol, or mixtures thereof.

Antioxidants include tocopherols and the esters thereof, sesamol of sesame oil, coniferyl benzoate of benzoin resin, nordihydroguaietic resin and nordihydroguaiaretic acid, gallates, butylated hydroxyanisole, ascorbic acid and salts and esters thereof, such as acorbyl palmitate, erythorbinic acid and salts and esters thereof, monothioglycerol, sodium formaldehyde sulfoxylate, sodium metabisulfite, sodium bisulfite, sodium sulfite, potassium metabisulfite, butylated hydroxy toluene, propionic acid, and combinations thereof.

Stabilizers include calcium chloride, calcium disodium ethylene diamine tetra-acetate, glucono delta-lactone, sodium gluconate, potassium gluconate, sodium tripolyphosphate, sodium hexametaphosphate, as cyclodextrins, cyclomannins (5 or more α-D-mannopyranose units linked at the 1,4 positions by α linkages), cyclogalactins (5 or more β-D-galactopyranose units linked at the 1,4 positions by β linkages), cycloatrins (5 or more α-D-altropyranose units linked at the 1,4 positions by a linkages), and combinations thereof.

Suitable coloring agents include azo dyes, organic or inorganic pigments, such as the oxides or iron or titanium, or coloring agents of natural origin. Anti-foaming and/or de-foaming agents include simethicone and similar agents.

Plasticizers include polyethylene glycols, polypropylene glycols, polyethylene-propylene glycols, glycerol, glycerol monoacetate, diacetate or triacetate, triacetin, polysorbate, cetyl alcohol, propylene glycol, sorbitol, sodium diethylsulfosuccinate, triethyl citrate, tributyl citrate, phytoextracts, fatty acid esters, fatty acids, and mixtures thereof.

Binders include starches, natural gums, pregelatinized starches, gelatin, polyvinylpyrrolidone, methylcellulose, sodium carboxymethylcellulose, ethylcellulose, polymerylamides, polyvinyloxoazolidone, polyvinyl alcohols, and mixtures thereof.

In some embodiments, pH modifiers are used to enhance the bioavailability of epinephrine, when epinephrine is included among the active pharmaceutical ingredients used. Such pH modifiers include weak organic acids including amino acids, phosphoric acid, acidic polymers, and salts and mixtures thereof. Weak organic acids include citric acid, acetic acid, succinic acid, tartaric acid, maleic acid, malic acid, ascorbic acid, acetylsalicylic acid, adipic acid, fumaric acid, glutaric acid, glutamic acid, itaconic acid, aspartic acid, lactic acid, and salts and mixtures thereof. Salts of acids include inorganic salts, such as alkali metal salt, alkaline earth metal salt, ammonium salt, and salts formed with an organic base, such as lysine, arginine and meglumine. In some embodiments, the pH modifiers are acidic polymers which serve the dual role of forming the polymeric layer and pH modification. Suitable acidic polymers include carboxymethylcellulose, cross-linked carboxymethylcellulose, poly(acrylic acid) polymers and copolymers, alginic acid, sodium alginate, calcium alginate, hyaluronic acid, mixtures and salts thereof.

Preparation Methods

The film compositions of invention may be prepared by conventional methods, such as solvent casting, hot melt extrusion, spinning, electro spinning, 3-dimensional or flexographic printing, spraying, electrospraying, combination of solvent casting and spraying, a combination of electrospinning and electrospraying, or any other permutation or combination of these methods. For example, methods described in Boda et al., J Aerosol Sci. 2018 November; 125: 164-181; Nguyen et al., J. Parm. Sci. 2016; 105: 2601-2620; Soares et al., Mater. Sci. Eng. C 2018 November; 92(1): 969-982; and JanBen et al., Int. J. Pharm. 2013 January; 441(1-2): 818-825 may be used.

In some embodiments, micronized particles of one or more of the active pharmaceutical ingredients are used.

The film may be formulated as a dispersion of active pharmaceutical ingredients in a polymeric solution, such as by solvent casting method, or epinephrine particles are deposited into/onto the polymer layer by alternative methods, such as electrospining, electrospraying, hot-melt extrusion, printing techniques (e.g., flexographic printing) or spraying, combination of solvent casting and spraying, solution blow spinning, electroblowing, centrifugal spinning, a combination of electrospinning and electrospraying, or any other suitable permutation or combination thereof.

Polymeric micro/nanofibers are produced by electrospinning, or other alternative methods (e.g. solution blowing, spinning method). The active pharmaceutical ingredients particles are deposited using alternative spraying methods, such as electrospraying, simultaneously, or consecutively when producing polymeric fibers.

In case of a multi-layered film compositions, the layers may be manufactured separately and then laminated or layered in succession. Binding solution of polymer(s) may be sprayed between two film layers, such as the polymeric and backing layer to combine the layers. The binding solution can contain excipients, such as mucoadhesive polymer, pH modifier, and/or absorption enhancer, thereby forming the third layer of a three-layered system in situ.

In some embodiments, in case of a multi-layered film, the polymeric layer comprising the active pharmaceutical ingredients is printed onto the mucoadhesive and/or backing layer, such as by employing a 3-dimensional or flexographic printing method. The mucoadhesive and/or backing layers may also be prepared by 3-dimensional or flexographic printing.

In some embodiments, the preparation method comprises:
providing a suspension or solution of one or more active pharmaceutical ingredients in a solvent/dispersant;
simultaneously or separately providing a suspension or solution of the polymer in a solvent/dispersant;
combining the active ingredient suspension/sloution and the polymer suspension/solution if prepared separately; and
removing the solvent/dispersant to obtain the polymeric layer.

Solvents/dispersants are used in the manufacturing process of oromucosal films of the invention to dissolve the active pharmaceutical ingredients, polymers and pharmaceutical excipients including plasticizers, pH modifiers, stabilizers, antioxidants, taste masking agents and absorption enhancers. All or just some of these excipients may be used depending on the requirements of the film formulation. In some embodiments, one or more the active pharmaceutical ingredients may remain suspended/dispersed or partially dissolved. In some embodiments, one or more polymers may remain suspended/dispersed or partially dissolved. In some embodiments, one or more excipients may remain suspended/dispersed or partially dissolved.

Suitable solvent/dispersant used in the preparation methods of the invention include lower alkyl alcohols, acetone, methyl acetate, ethyl acetate, isopropyl acetate, acetonitrile, heptane, tetrahydrofuran, water, alkaline water, aqueous buffers, and mixtures thereof. Suitable lower alkyl alcohols include methanol, ethanol, isopropyl alcohol, 2-propanol, 1-propanol 1-butanol, t-butanol, and mixtures thereof.

During the manufacturing process, those solvents/dispersants may be used when processing active ingredient particles that do not dissolve the particles to any appreciable extent. The solvents/dispersant should disperse solid particles without substantial dissolution to ensure the vast majority of the active ingredient is maintained as particulate in nature without any recrystalinization, degradation or oxidization. More specificaly, the active ingredient particles are insoluble, practically insoluble, very slightly soluble or slightly soluble (as defined in the United States Pharmacopeia) in the solvents/dispersant used when a film comprising particles of the active ingredient is desired.

In some embodiments an aqueous dispersion of epinephrine base solid particles is prepared, and the pH of the dispersion is maintained above about 8 using a base or an alkali to prevent dissolution of the particles. For example, the pH of the dispersion may be maintained between about 8.5 to about 9.5.

Use

The film compositions of the invention may be used for providing anesthesia, or for the treatment of diabetes, epilepsy, tooth pain, mouth ulcers, cardiac arrest, asthma, bronchial asthma, bronchitis, emphysema, respiratory infections, anaphylactic shock, or allergic reactions in a subject in need thereof. The composition may be administered sublingually, buccally or via any other suitable mucosal surface.

When a local anesthetic is included, the film compositions of the invention may be used for the providing for local anesthesia, such as for treatment and/or prevention of tooth pain. A lower dose of epinephrine is used in combination with local anesthetics, such as lodicaine, novocaine, articaine, prilocaine, or mepivacaine, in a slower dissolving polymer for local or dental anaesthesia. The amount of epinephrine used in such cases may be from about 0.005% to about 5%, from about 0.1% to about 2%, from about 0.25% to about 1%, or from about 0.5% to about 1%. The amount of local anesthetic used in such cases may be from about 5% to about 90%, from about 10% to about 85%, or from about 50% to about 80%, or from about 50% to about 60%. The amount of polymer used in the polymeric layer may be from about 5% to about 40%, from about 10% to about 35%, or from about 15% to about 30. The amount of plasticizer used in the polymeric layer may be from about 1% to about 10%, from about 2% to about 8%, from about 2% to about 5%, or from about 2% to about 3.

In addition to providing drug delivery, once the film composition adheres to the mucosal surface, it may also provide protection to the treatment site by acting as an erodible bandage.

### EXAMPLES

The examples demonstrate that the active pharmaceutical ingredients formulated using methods of the invention are compatible with each other, with absorption enhancers, and other excipients when formulated into film compositions of the invention. The examples also show that the active ingredient is released rapidly from the film compositions of the invention and is quickly and effectively absorbed, as demonstrated under both ex-vivo and in-vivo conditions.

### Example 1

### Preparation of the Oromucosal Film Composition of the Invention

Various formulations were designed including single, bi and tri-layered oromucosal films to demostrate the use of API in alternative formulation designs. Selected solvents/dispersants as listed previously (page 14) were used in the formulation of the API-containing layer (active layer).

The Preparation Method

Each layer was formed separately (in the case of bi- and tri-layered films) and then laminated/combined to form the desired oromucosal film compositions. Alternatively, the layers could be built up in succession using the solvent casting or flexographic printing technique.

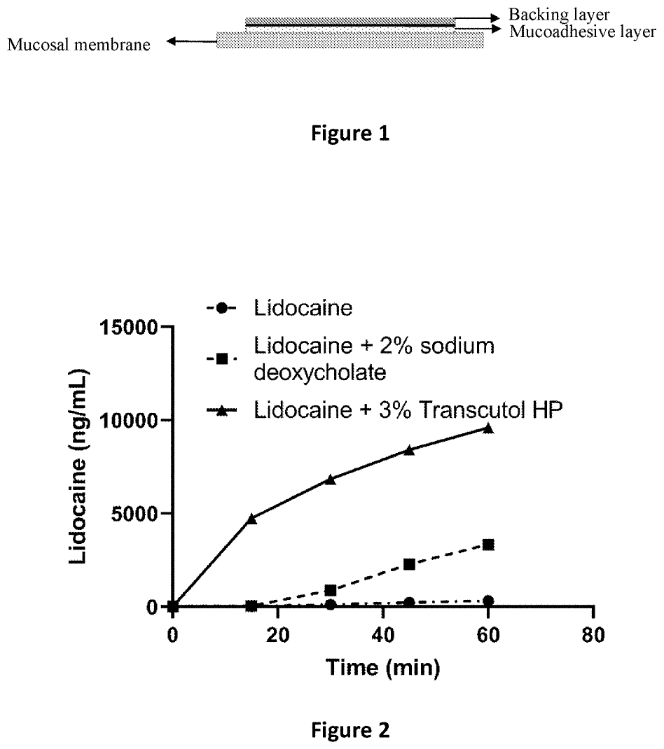

Each bi-layered film consists of 1) a backing layer and 2) a mucoadhesive active layer (FIG. 1). The upper surface of a prepared mucoadhesive layer was coated with a polymer to form: i) a non-soluble backing layer using ethylcellulose (Eleftheriadis et al. Fabrication of Mucoadhesive Buccal Films for Local Administration of Ketoprofen and Lidocaine Hydrochloride by Combining Fused Deposition Modeling and Inkjet Printing. J Pharm Sci. 2020 September; 109(9):2757-2766. doi: 10.1016/j.xphs.2020.05.022) or polycaprolactone (Colley et al. Pre-clinical evaluation of novel mucoadhesive bilayer patches for local delivery of clobetasol-17-propionate to the oral mucosa. Biomaterials. 2018 September; 178:134-146. doi: 10.1016/j.biomaterials.2018.06.009, Edmans et al. Mucoadhesive Electrospun Fibre-Based Technologies for Oral Medicine. Pharmaceutics. 2020 Jun. 2; 12(6):504. doi: 10.3390/pharmaceutics12060504); or ii) soluble backing layer (eg amino methacrylate copolyme-Eudragit®(Magek et al. Multi-layered nanofibrous mucoadhesive films for buccal and sublingual administration of drug-delivery and vaccination nanoparticles—important step towards effective mucosal vaccines. J Control Release. 2017 Mar. 10; 249:183-195. doi: 10.1016/j.jconrel.2016.07.036)).

A variety of polymers, mucoadhesive polymers, and pharmaceutical excipients, including pH modifiers, absorption enhancers and antioxidants couldmay be used in the formulations. Many other alternative absorption enhancers, plasticizers or anti-oxidants etc may be used.

The non-adhesive backing layer was formed by i) spraying a 2.5% ethanolic solution of ethylcellulose (Ethocel™, Colorcon Limited, UK) with 2% dibutyl phthalate (DBP, Merck KGaA, Germany) directly onto the surface of the mucoadhesive layer API-containig layer, or ii) by spraying an ethanolic solution of amino methacrylate copolymer (Eudragit®L100-55, Evonik, Germany) to form an oro-dissolving backing layer (Magek et al. Multi-layered nanofibrous mucoadhesive films for buccal and sublingual administration of drug-delivery and vaccination nanoparticles—important step towards effective mucosal vaccines. J Control Release. 2017 Mar. 10; 249:183-195. doi: 10.1016/j.jconrel.2016.07.036). The non-adhesive backing layer was alternatively formed using solvent casting methodology and laminated to the mucoadhesive layer or API-containing layer.

Active layers (polymer or mucoadhesive layer comprising API) were prepared using selected solvents/dispersants and polymers:

Hypromellose 2910 (hydroxypropyl methylcellulose, viscosity 6 mPa·s, Pharmacoat® 606, Shin-Etsu Chemical Co. Ltd., Japan) and hydroxypropyl cellulose (viscosity 300-600 mPa·s, Klucel™ EF PHARM, Ashland, USA) in a 2:1 (w/w) ratio were added to the chosen solvent/disperant (ethanol, water, alkaline water of pH above 8.5 or mixture thereof). Glycerol (Merck KGaA, Germany) at a concentration 3-7% (w/w) was added as a plasticizer. Sodium metabisulfite (Merck KGaA, Germany) in the concentration 0.16-0.24% was added as an antioxidant to stabilize the solution. Epinephrine base particles (Cambrex Profarmaco Milano S.r.l., Italy) were added at a concentration of 1.2-1.8% (w/w). Diethylene glycol monoethyl ether (Transcutol® HP, Gattefosse, France), sodium deoxycholate (Merck KGaA, Germany), caprylocaproyl polyoxyl-8 glycerides NF (Labrasol®, Gattefosse, France), eugenol (Merck KGaA, Germany), disodium ethylenediaminetetraacetic acid (EDTA Na, Merck KGaA, Germany) were added as absorption enhancers (3-13% w/w). The wet mixtures were then treated by sonication to remove air bubbles and the required volumes poured out into Petri dishes. The solvent/dispersant was then removed by evaporation at a temperature of 37° C. or 60° C. (ethanol, water, alkaline water of pH above 8.5).

The following tables describes examples of the composition of the active layers:

I. Lidocaine Hydrochloride

TABLE Ia
Mucoadhesive layer prepared by solvent casting method (polymer
swelling/dissolution conditions: 3 hrs at 25° C.; solvent
evaporation: 24 hrs at 37° C.) using water 0.331 ml/cm2:

| Material | % of dry composition | mg/cm² film |
|---|---|---|
| Lidocaine Hydrochloride | 38.72 | 5.00 |
| Pharmacoat ® 606 | 37.44 | 4.84 |
| Klucel ™ EF | 18.72 | 2.42 |
| Glycerol | 5.13 | 0.66 |
| Total | 100.00 | 12.92 |

TABLE Ib
Mucoadhesive layer prepared by solvent casting method
(polymer swelling/dissolution conditions: 3 hrs at
25° C.; solvent evaporation: 4 hrs at 37°
C.) using ethanol and water (80/20% v/v) 0.331 ml/cm2:

| Material | % of dry composition | mg/cm² film |
|---|---|---|
| Lidocaine Hydrochloride | 39.02 | 5.00 |
| Pharmacoat ® 606 | 41.86 | 5.37 |
| Klucel ™ EF | 13.95 | 1.79 |
| Glycerol | 5.17 | 0.66 |
| Total | 100.00 | 12.82 |

TABLE Ic
Mucoadhesive layer prepared by solvent casting method
(polymer swelling/dissolution conditions: 3 hrs at
25° C.; solvent evaporation: 4 hrs at 37°
C.) using ethanol and water (80/20% v/v) 0.331 ml/cm2:

| Material | % of dry composition | mg/cm² film |
|---|---|---|
| Lidocaine Hydrochloride | 55.35 | 10.00 |
| Pharmacoat ® 606 | 29.40 | 5.37 |
| Klucel ™ EF | 9.80 | 1.79 |
| Glycerol | 5.44 | 0.99 |
| Total | 100.00 | 18.15 |

II. Lidocaine Hydrochloride with Permeation Enhancers

a) Transcutol® HP

TABLE IIa
Mucoadhesive layer prepared by solvent casting method (polymer
swelling/dissolution conditions: 3 hrs at 25° C.; solvent
evaporation: 24 hrs at 37° C.) using water 0.331 ml/cm²:

| Material | % of dry composition | mg/cm² film |
|---|---|---|
| Lidocaine Hydrochloride | 36.39 | 5.00 |
| Pharmacoat ® 606 | 35.18 | 4.84 |
| Klucel ™ EF | 17.59 | 2.42 |
| Transcutol ® HP | 6.02 | 0.83 |
| Glycerol | 4.82 | 0.66 |
| Total | 100.00 | 13.75 |

TABLE IIb
Mucoadhesive layer prepared by solvent casting method (polymer
swelling/dissolution conditions: 3 hrs at 25° C.; solvent
evaporation: 3 hrs at 60° C.) using water 0.331 ml/cm²:

| Material | % of dry composition | mg/cm² film |
|---|---|---|
| Lidocaine Hydrochloride | 33.78 | 5.00 |
| Pharmacoat ® 606 | 32.66 | 4.84 |
| Klucel ™ EF | 16.33 | 2.42 |
| Transcutol ® HP | 10.51 | 1.56 |
| Glycerol | 6.71 | 0.99 |
| Total | 100.00 | 14.81 |

TABLE IIc
Mucoadhesive layer prepared by solvent casting method
(polymer swelling/dissolution conditions: 3 hrs at
25° C.; solvent evaporation: 2 hrs at 60° C.)
using ethanol and water (80/20% v/v) 0.331 ml/cm²:

| Material | % of dry composition | mg/cm² film |
|---|---|---|
| Lidocaine Hydrochloride | 37.28 | 5.00 |
| Pharmacoat ® 606 | 36.05 | 4.84 |
| Klucel ™ EF | 18.02 | 2.42 |
| Transcutol ® HP | 3.21 | 0.43 |
| Glycerol | 5.43 | 0.73 |
| Total | 100.00 | 13.42 |

TABLE IId
Mucoadhesive layer prepared by solvent casting method
(polymer swelling/dissolution conditions: 3 hrs at
25° C.; solvent evaporation: 2 hrs at 60° C.)
using ethanol and water (80/20% v/v) 0.331 ml/cm²:

| Material | % of dry composition | mg/cm² film |
|---|---|---|
| Lidocaine Hydrochloride | 36.65 | 5.00 |
| Pharmacoat ® 606 | 39.32 | 5.37 |
| Klucel ™ EF | 13.11 | 1.79 |
| Transcutol ® HP | 6.07 | 0.83 |
| Glycerol | 4.85 | 0.66 |
| Total | 100.00 | 13.65 |

TABLE IIe
Mucoadhesive layer prepared by solvent casting method
(polymer swelling/dissolution conditions: 3 hrs at
25° C.; solvent evaporation: 2 hrs at 60° C.)
using ethanol and water (80/20% v/v) 0.331 ml/cm²:

| Material | % of dry composition | mg/cm² film |
|---|---|---|
| Lidocaine Hydrochloride | 33.78 | 5.00 |
| Pharmacoat ® 606 | 32.66 | 4.84 |
| Klucel ™ EF | 16.33 | 2.42 |
| Transcutol ® HP | 10.51 | 1.56 |
| Glycerol | 6.71 | 0.99 |
| Total | 100.00 | 14.81 |

TABLE IIf
Mucoadhesive layer prepared by solvent casting method
(polymer swelling/dissolution conditions: 3 hrs at
25° C.; solvent evaporation: 4 hrs at 37° C.)
using ethanol and water (80/20% v/v) 0.331 ml/cm²:

| Material | % of dry composition | mg/cm² film |
|---|---|---|
| Lidocaine Hydrochloride | 36.65 | 5.00 |
| Pharmacoat ® 606 | 39.32 | 5.37 |
| Klucel ™ EF | 13.11 | 1.79 |
| Transcutol ® HP | 6.07 | 0.83 |
| Glycerol | 4.85 | 0.66 |
| Total | 100.00 | 13.65 |

TABLE IIg
Mucoadhesive layer prepared by solvent casting method
(polymer swelling/dissolution conditions: 3 hrs at
25° C.; solvent evaporation: 4 hrs at 37° C.)
using ethanol and water (80/20% v/v) 0.331 ml/cm²:

| | % of dry | |
|---|---|---|
| Material | composition | mg/cm² film |
| Lidocaine Hydrochloride | 35.20 | 5.00 |
| Pharmacoat ® 606 | 37.76 | 5.37 |
| Klucel ™ EF | 12.59 | 1.79 |
| Transcutol ® HP | 9.79 | 1.39 |
| Glycerol | 4.66 | 0.66 |
| Total | 100.00 | 14.21 |

TABLE IIh
Mucoadhesive layer prepared by solvent casting method
(polymer swelling/dissolution conditions: 3 hrs at
25° C.; solvent evaporation: 6 hrs at 37° C.)
using ethanol and water (50/50% v/v) 0.331 ml/cm²:

| | % of dry | |
|---|---|---|
| Material | composition | mg/cm² film |
| Lidocaine Hydrochloride | 47.58 | 10.10 |
| Pharmacoat ® 606 | 25.27 | 5.37 |
| Klucel ™ EF | 8.42 | 1.79 |
| Transcutol ® HP | 12.48 | 2.65 |
| Glycerol | 6.24 | 1.33 |
| Total | 100.00 | 21.24 |

b) Sodium Deoxycholate

TABLE IIi
Mucoadhesive layer prepared by solvent casting method
(polymer swelling/dissolution conditions: 3 hrs at
25° C.; solvent evaporation: 2 hrs at 60° C.)
using ethanol and water (80/20% v/v) 0.331 ml/cm²:

| | % of dry | |
|---|---|---|
| Material | composition | mg/cm² film |
| Lidocaine Hydrochloride | 37.28 | 5.00 |
| Pharmacoat ® 606 | 36.05 | 4.84 |
| Klucel ™ EF | 18.02 | 2.42 |
| Sodium deoxycholate | 3.21 | 0.43 |
| Glycerol | 5.43 | 0.73 |
| Total | 100.00 | 13.42 |

c) Labrasol

TABLE IIj
Mucoadhesive layer prepared by solvent casting method
(polymer swelling/dissolution conditions: 3 hrs at
25° C.; solvent evaporation: 2 hrs at 60° C.)
using ethanol and water (80/20% v/v) 0.331 ml/cm²:

| | % of dry | |
|---|---|---|
| Material | composition | mg/cm² film |
| Lidocaine Hydrochloride | 37.28 | 5.00 |
| Pharmacoat ® 606 | 36.05 | 4.84 |
| Klucel ™ EF | 18.02 | 2.42 |
| Labrasol | 3.21 | 0.43 |
| Glycerol | 5.43 | 0.73 |
| Total | 100.00 | 13.42 |

d) Disodium Ethylenediaminetetraacetic Acid Salt

TABLE IIk
Mucoadhesive layer prepared by solvent casting method
(polymer swelling/dissolution conditions: 3 hrs at
25° C.; solvent evaporation: 2 hrs at 60° C.)
using ethanol and water (80/20% v/v) 0.331 ml/cm²:

| | % of dry | |
|---|---|---|
| Material | composition | mg/cm² film |
| Lidocaine Hydrochloride | 37.28 | 5.00 |
| Pharmacoat ® 606 | 36.05 | 4.84 |
| Klucel ™ EF | 18.02 | 2.42 |
| EDTA Na | 3.21 | 0.43 |
| Glycerol | 5.43 | 0.73 |
| Total | 100.00 | 13.42 |

e) Eugenol

TABLE IIl
Mucoadhesive layer prepared by solvent casting method
(polymer swelling/dissolution conditions: 3 hrs at
25° C.; solvent evaporation: 2 hrs at 60° C.)
using ethanol and water (80/20% v/v) 0.331 ml/cm²:

| | % of dry | |
|---|---|---|
| Material | composition | mg/cm² film |
| Lidocaine Hydrochloride | 36.12 | 5.00 |
| Pharmacoat ® 606 | 34.93 | 4.84 |
| Klucel ™ EF | 17.46 | 2.42 |
| Eugenol | 6.22 | 0.86 |
| Glycerol | 5.26 | 0.73 |
| Total | 100.00 | 13.85 |

f) Transcutol® HP+Eugenol

TABLE IIm
Mucoadhesive layer prepared by solvent casting method
(polymer swelling/dissolution conditions: 3 hrs at
25° C.; solvent evaporation: 2 hrs at 60° C.)
using ethanol and water (80/20% v/v) 0.331 ml/cm²:

| | % of dry | |
|---|---|---|
| Material | composition | mg/cm² film |
| Lidocaine Hydrochloride | 32.13 | 5.00 |
| Pharmacoat ® 606 | 31.06 | 4.84 |
| Klucel ™ EF | 15.53 | 2.42 |
| Eugenol | 6.17 | 0.96 |
| Transcutol ® HP | 10.00 | 1.56 |
| Glycerol | 5.11 | 0.80 |
| Total | 100.00 | 15.57 |

III. Lidocaine Hydrochloride, Prilocaine Hydrochloride

TABLE III
Mucoadhesive layer prepared by solvent casting method
(polymer swelling/dissolution conditions: 3 hrs at
25° C.; solvent evaporation: 4 hrs at 37° C.)
using ethanol and water (80/20% v/v) 0.331 ml/cm²:

| | % of dry | |
|---|---|---|
| Material | composition | mg/cm² film |
| Lidocaine Hydrochloride | 27.55 | 5.00 |
| Prilocaine Hydrochloride | 27.55 | 5.00 |
| Pharmacoat ® 606 | 29.56 | 5.37 |
| Klucel ™ EF | 9.85 | 1.79 |
| Transcutol ® HP | 0.00 | 0.00 |
| Glycerol | 5.47 | 0.99 |
| Total | 100.00 | 18.15 |

IV. Lidocaine Hydrochloride, Prilocaine Hydrochloride and Permeation Enhancers

TABLE IVa
Mucoadhesive layer prepared by solvent casting method
(polymer swelling/dissolution conditions: 3 hrs at
25° C.; solvent evaporation: 4 hrs at 37° C.)
using ethanol and water (80/20% v/v) 0.331 ml/cm²:

| | % of dry | |
|---|---|---|
| Material | composition | mg/cm² film |
| Lidocaine Hydrochloride | 23.89 | 5.00 |
| Prilocaine Hydrochloride | 23.89 | 5.00 |
| Pharmacoat ® 606 | 25.63 | 5.37 |
| Klucel ™ EF | 8.54 | 1.79 |
| Transcutol ® HP | 13.29 | 2.78 |
| Glycerol | 4.75 | 0.99 |
| Total | 100.00 | 20.93 |

TABLE IVb
Mucoadhesive layer prepared by solvent casting method
(polymer swelling/dissolution conditions: 3 hrs at
25° C.; solvent evaporation: 4 hrs at 37° C.)
using ethanol and water (80/20% v/v) 0.331 ml/cm²:

| | % of dry | |
|---|---|---|
| Material | composition | mg/cm² film |
| Lidocaine Hydrochloride | 28.95 | 7.52 |
| Prilocaine Hydrochloride | 28.95 | 7.52 |
| Pharmacoat ® 606 | 20.66 | 5.37 |
| Klucel ™ EF | 6.89 | 1.79 |
| Transcutol ® HP | 10.71 | 2.78 |
| Glycerol | 3.83 | 0.99 |
| Total | 100.00 | 25.97 |

TABLE IVc
Mucoadhesive layer prepared by solvent casting method
(polymer swelling/dissolution conditions: 3 hrs at
25° C.; solvent evaporation: 4 hrs at 37° C.)
using ethanol and water (80/20% v/v) 0.331 ml/cm²:

| | % of dry | |
|---|---|---|
| Material | composition | mg/cm² film |
| Lidocaine Hydrochloride | 32.33 | 10.00 |
| Prilocaine Hydrochloride | 32.33 | 10.00 |
| Pharmacoat ® 606 | 17.34 | 5.37 |
| Klucel ™ EF | 5.78 | 1.79 |
| Transcutol ® HP | 8.99 | 2.78 |
| Glycerol | 3.21 | 0.99 |
| Total | 100.00 | 30.93 |

V. Lidocaine Hydrochloride, Epinephrine Base and Permeation Enhancers (Transcutol® and Eugenol)

TABLE V
Mucoadhesive layer prepared by solvent casting method
(polymer swelling/dissolution conditions: 3 hrs at
25° C.; solvent evaporation: 2 hrs at 60° C.) using
ethanol and alkaline water (80/20% v/v) 0.331 ml/cm²:

| | % of dry | |
|---|---|---|
| Material | composition | mg/cm² film |
| Lidocaine Hydrochloride | 31.49 | 5.00 |
| Pharmacoat ® 606 | 30.45 | 4.84 |
| Klucel ™ EF | 15.22 | 2.42 |
| Epinephrine base | 1.56 | 0.25 |
| Sodium metabisulfite | 0.21 | 0.03 |
| Transcutol ® HP | 10.01 | 1.59 |
| Eugenol | 6.05 | 0.96 |
| Glycerol | 5.01 | 0.80 |
| Total | 100.00 | 15.89 |

VI. Lidocaine Hydrochloride, Fluticasone Propionate with Permeation Enhancers

TABLE VIa
Mucoadhesive layer prepared by solvent casting method
(polymer swelling/dissolution conditions: 3 hrs at
25° C.; solvent evaporation: 24 hrs at 37° C.)
using ethanol and water (80/20% v/v) 0.331 ml/cm²:

| | % of dry | |
|---|---|---|
| Material | composition | mg/cm² film |
| Lidocaine Hydrochloride | 36.35 | 5.00 |
| Pharmacoat ® 606 | 35.15 | 4.84 |
| Klucel ™ EF | 17.57 | 2.42 |
| Fluticasone Propionate | 0.10 | 0.01 |
| Transcutol ® HP | 6.02 | 0.83 |
| Glycerol | 4.81 | 0.66 |
| Total | 100.00 | 13.76 |

TABLE VIb
Mucoadhesive layer prepared by solvent casting method
(polymer swelling/dissolution conditions: 3 hrs at
25° C.; solvent evaporation: 2 hrs at 60° C.)
using ethanol and water (80/20% v/v) 0.331 ml/cm²:

| | % of dry | |
|---|---|---|
| Material | composition | mg/cm² film |
| Lidocaine Hydrochloride | 33.43 | 5.00 |
| Pharmacoat ® 606 | 32.32 | 4.84 |
| Klucel ™ EF | 16.16 | 2.42 |
| Fluticasone Propionate | 0.38 | 0.05 |
| Eugenol | 11.07 | 1.66 |
| Glycerol | 6.64 | 0.99 |
| Total | 100.00 | 14.96 |

TABLE VIc
Mucoadhesive layer prepared by solvent casting method
(polymer swelling/dissolution conditions: 3 hrs at
25° C.; solvent evaporation: 2 hrs at 60° C.)
using ethanol and water (80/20% v/v) 0.331 ml/cm²:

| | % of dry | |
|---|---|---|
| Material | composition | mg/cm² film |
| Lidocaine Hydrochloride | 30.28 | 5.00 |
| Pharmacoat ® 606 | 29.28 | 4.84 |
| Klucel ™ EF | 14.64 | 2.42 |
| Fluticasone Propionate | 0.34 | 0.05 |
| Eugenol | 10.03 | 1.66 |
| Transcutol ® HP | 9.42 | 1.56 |
| Glycerol | 6.02 | 0.99 |
| Total | 100.00 | 16.52 |

### Example II

### The Effect of Permeation Enhancers on Permeation of Lidocaine Hydrochloride Dissolved in the Solution Ex Vivo

The objective of this study was to prove the effect of absorption enhancers on the absorption rate of dissolved lidocaine hydrochloride through excised sublingual porcine mucosa ex vivo using a Franz diffusion cell (PermeGear, SES GmbH Analysesysteme, Germany).

### Methods

Porcine sublingual mucosa was removed immediately after sacrifice. The mucosa was then prepared and mounted to the diffusion cell with the receptor compartment having a volume of 5 mL, containing 0.9% NaCl solution. The volume of 280 μl of the solution of lidocaine HCl, or lidocaine HCl with various permeation enhancers were tested. The concentration of lidocaine HCl (189 mg/ml) in the donor compartment corresponded to 153 mg/mL of lidocaine.

The permeation rate was assessed by measuring the increasing concentration of lidocaine in the receptor compartment under constant mixing at 37° C. Samples were taken at 0, 15, 30, 45, 60, 75, 90, 115 and 120 minutes to measure the concentration of Lidocaine in the receptor chamber. The concentration level was determined using HPLC/MS method.

TABLE VIIa
The following solutions of lidocaine HCl were tested:
Composition of sample: Lidocaine HCl 189 mg/ml (Lidocaine 153 mg/ml)

| | |
|---|---|
| 1 | Lidocaine HCl |
| 2 | Lidocaine HCl + Transcutol ® HP 3% |
| 3 | Lidocaine HCl + Sodium deoxycholate 2% |
| 4 | Lidocaine HCl + Labrasol 6% |
| 5 | Lidocaine HCl + EDTA disodium salt 3%, 6% |

### Results

Significantly increased permeation of lidocaine through sublingual mucosa ex vivo was observed within minutes when combined with Transcutol® HP, Sodium deoxycholate, Labrasol and EDTA disodium salt as permeation enhancers.

TABLE VIIb
The permeation of Lidocaine through sublingual mucosa ex vivo

| | | PERMEATION ENHANCER TESTED (μg/mL Lidocaine permeated) | | | | |
|---|---|---|---|---|---|---|
| Time | without permeation | Transcutol ® | Sodium | Labrasol | EDTA disodium | EDTA disodium |
| (min) | enhancers | HP 3% | deoxycholate 2% | 6% | salt 3% | salt 6% |
| 0 | 3.1 | 6.9 | 36.6 | 0.0 | 0.0 | 0.0 |
| 15 | 29.9 | 4553.2 | 44.0 | 233.8 | 57.4 | 154.1 |
| 30 | 111.5 | 6306.3 | 815.2 | 236.7 | 142.0 | 278.4 |
| 45 | 201.4 | 7442.3 | 2021.3 | 184.1 | 239.5 | 420.6 |
| 60 | 269.9 | 8145.0 | 2841.5 | 184.3 | 366.5 | 538.7 |
| 75 | 313.8 | 3050.8 | 3736.3 | 2121.4 | 441.7 | 620.1 |
| 90 | 372.1 | 4588.6 | 4272.5 | 2486.0 | 496.4 | 677.8 |
| 105 | 429.9 | 9587.7 | 4550.9 | 2987.4 | 540.4 | 794.0 |
| 120 | 463.7 | 9706.9 | 4922.6 | 3342.8 | 595.9 | 889.8 |

### Conclusion

FIG. 2 shows the levels of lidocaine permeated through oral (sublingual) mucosa ex vivo. A nearly linear increase in levels of permeated lidocaine (FIG. 2) was observed in the time frame of 120 minutes. FIG. 2 shows the influence of absorption enhancers on the permeation rate using Franz diffusion cell. It was demonstrated that the permeation rate of lidocaine was significantly increased when combined with permeation enhancers.

### Example III

### The Effect of Permeation Enhancers on Permeation of Lidocaine Hydrochloride and Prilocaine Hydrochloride Formulated as a Mucoadhesive Oral Film Ex Vivo

The objective of this study was to prove the effect of absorption enhancers on the absorption rate of lidocaine hydrochloride and prilocaine hydrochloride formulated as a mucoadhesive oral film of the invention following the application of the film onto excised sublingual porcine mucosa ex-vivo using a Franz diffusion cell model (PermeGear, SES GmbH Analysesysteme, Germany).

### Methods

Porcine sublingual mucosa was removed immediately after sacrifice. The mucosa was then prepared and mounted to the diffusion cell with the receptor compartment having a volume of 12 mL, containing 0.9% NaCl solution

The following film were tested:
Lidocaine HCl
Lidocaine HCl with various permeation enhancers
Lidocaine HCl and Prilocaine HCl
Lidocaine HCl and Prilocaine HCl with permeation enhancers

The permeation rate was assessed by measuring the increasing concentration of lidocaine and prilocaine in the receptor compartment under constant mixing at 37° C. The volume of 290 μl of samples were taken at 0, 15, 30, 45, 60, 75, 90, 115 and 120 minutes to measure the concentration of lidocaine/prilocaine in the receptor chamber. The concentration level was determined using HPLC/MS method.

### Preparation of Mucoadhesive Film

The mucoadhesive film was formulated as a bi-layered film containing i) 6 mg of lidocaine HCl/film, or ii) lidocaine HCl 6 mg and prilocaine HCl 6 mg/film with or without permeation enhancers. Non-adhesive, non-soluble backing layers were formed by the spraying of a 2.5% ethanolic solution ofeEthylcellulose (Ethocel™, Colorcon Limited, UK) with 2% Dibutyl phthalate (DBP, Merck KGaA, Germany) directly onto the surface of a mucoadhesive layer.

Mucoadhesive layer was formulated by solvent casting method (polymer swelling/dissolution conditions: 2 hrs at 25° C.; solvent evaporation: 2 hrs at 60° C.) using ethanol and water (80/20% v/v) 0.331 ml/cm². The following formulations of lidocaine/prilocaine hydrochloride in mucoadhesive films were tested:

Formulation F1

| | % of dry | |
|---|---|---|
| Material | composition | mg/cm² film |
| Lidocaine Hydrochloride | 38.72 | 5.00 |
| Pharmacoat ® 606 | 37.44 | 4.84 |
| Klucel ™ EF | 18.72 | 2.42 |
| Glycerol | 5.13 | 0.66 |
| Total | 100.00 | 12.92 |

Formulation F2

| | % of dry | |
|---|---|---|
| Material | composition | mg/cm² film |
| Lidocaine Hydrochloride | 34.63 | 5.00 |
| Pharmacoat ® 606 | 33.49 | 4.84 |
| Klucel ™ EF | 16.74 | 2.42 |
| Transcutol ® HP | 10.09 | 1.46 |
| Glycerol | 5.05 | 0.73 |
| Total | 100.00 | 14.45 |

Formulation F3

| | % of dry | |
|---|---|---|
| Material | composition | mg/cm² film |
| Lidocaine Hydrochloride | 37.28 | 5.00 |
| Pharmacoat ® 606 | 36.05 | 4.84 |
| Klucel ™ EF | 18.02 | 2.42 |
| Sodium deoxycholate | 3.21 | 0.43 |
| Glycerol | 5.43 | 0.73 |
| Total | 100.00 | 13.42 |

Formulation F4

| | % of dry | |
|---|---|---|
| Material | composition | mg/cm² film |
| Lidocaine Hydrochloride | 37.28 | 5.00 |
| Pharmacoat ® 606 | 36.05 | 4.84 |
| Klucel ™ EF | 18.02 | 2.42 |
| Labrasol | 3.21 | 0.43 |
| Glycerol | 5.43 | 0.73 |
| Total | 100.00 | 13.42 |

Formulation F5

| | % of dry | |
|---|---|---|
| Material | composition | mg/cm² film |
| Lidocaine Hydrochloride | 37.28 | 5.00 |
| Pharmacoat ® 606 | 36.05 | 4.84 |
| Klucel ™ EF | 18.02 | 2.42 |
| EDTA Na | 3.21 | 0.43 |
| Glycerol | 5.43 | 0.73 |
| Total | 100.00 | 13.42 |

Formulation F6

| | % of dry | |
|---|---|---|
| Material | composition | mg/cm² film |
| Lidocaine Hydrochloride | 36.12 | 5.00 |
| Pharmacoat ® 606 | 34.93 | 4.84 |
| Klucel ™ EF | 17.46 | 2.42 |
| Eugenol | 6.22 | 0.86 |
| Glycerol | 5.26 | 0.73 |
| Total | 100.00 | 13.85 |

Formulation F7

| | % of dry | |
|---|---|---|
| Material | composition | mg/cm² film |
| Lidocaine Hydrochloride | 32.13 | 5.00 |
| Pharmacoat ® 606 | 31.06 | 4.84 |
| Klucel ™ EF | 15.53 | 2.42 |
| Eugenol | 6.17 | 0.96 |
| Transcutol ® HP | 10.00 | 1.56 |
| Glycerol | 5.11 | 0.80 |
| Total | 100.00 | 15.57 |

Formulation F8

| | % of dry | |
|---|---|---|
| Material | composition | mg/cm² film |
| Lidocaine Hydrochloride | 27.50 | 5.00 |
| Prilocaine Hydrochloride | 27.50 | 5.00 |
| Pharmacoat ® 606 | 26.59 | 4.84 |
| Klucel ™ EF | 13.30 | 2.42 |
| Glycerol | 5.10 | 0.93 |
| Total | 100.00 | 18.19 |

Formulation F9

| | % of dry | |
|---|---|---|
| Material | composition | mg/cm² film |
| Lidocaine Hydrochloride | 24.59 | 5.00 |
| Prilocaine Hydrochloride | 24.59 | 5.00 |
| Pharmacoat ® 606 | 23.78 | 4.84 |
| Klucel ™ EF | 11.89 | 2.42 |
| Transcutol ® HP | 10.10 | 2.05 |
| Glycerol | 5.05 | 1.03 |
| Total | 100.00 | 20.34 |

Formulation F10

| | % of dry | |
|---|---|---|
| Material | composition | mg/cm² film |
| Lidocaine Hydrochloride | 22.88 | 5.00 |
| Prilocaine Hydrochloride | 22.88 | 5.00 |
| Pharmacoat ® 606 | 22.12 | 4.84 |
| Klucel ™ EF | 11.06 | 2.42 |
| Eugenol | 6.06 | 1.33 |
| Transcutol ® HP | 10.00 | 2.19 |
| Glycerol | 5.00 | 1.09 |
| Total | 100.00 | 21.87 |

TABLE VIII
Composition of sample: Lidocaine HCl (6 mg/film)

| | |
|---|---|
| F1 | Lidocaine HCl |
| F2 | Lidocaine HCl + Transcutol HP 10% |
| F3 | Lidocaine HCl + Sodium deoxycholate 3% |
| F4 | Lidocaine HCl + Labrasol 3% |
| F5 | Lidocaine HCl + EDTA disodium salt 3% |
| F6 | Lidocaine HCl + Eugenol 6% |
| F7 | Lidocaine HCl + Transcutol 10% + Eugenol 6% |

Composition of sample: Lidocaine HCl + Prilocaine HCl (6 + 6 mg/film)

| | |
|---|---|
| F8 | Prilocaine HCl + Lidocaine HCl |
| F9 | Prilocaine HCl + Lidocaine HCl + Transcutol 10% |
| F10 | Prilocaine HCl + Lidocaine HCl + Transcutol 10% + Eugenol 6% |

### Results

Significantly increased permeation of i) lidocaine and ii) lidocaine and prilocaine from mucoadhesive bi-layered film through sublingual mucosa ex vivo were observed within minutes when combined with Transcutol® HP, Sodium deoxycholate, labrasol®, EDTA disodium salt and eugenol or combination of Transcutol® HP and eugenol as permeation enhancers.

TABLE IX
The permeation of Lidocaine from mucoadhesive film
(sample F1-F7) through sublingual mucosa ex vivo

| | | PERMEATION ENHANCER TESTED (μg/mL Lidocaine permeated) | | | | | |
|---|---|---|---|---|---|---|---|
| | without | | | | EDTA | | Transcutol |
| Time | permeation | Transcutol | Sodium | Labrasol | disodium | Eugenol | 10% + |
| (min) | enhancers | HP 10% | deoxycholate 3% | 3% | salt 3% | 6% | Eugenol 6% |
| 0 | 1.6 | 0.0 | 0.0 | 0.0 | 0.0 | 8.1 | 2.1 |
| 15 | 13.6 | 559.5 | 737.7 | 21.1 | 40.4 | 173.7 | 153.5 |
| 30 | 27.3 | 648.3 | 468.9 | 56.0 | 71.0 | 336.7 | 373.0 |
| 45 | 42.9 | 714.8 | 593.8 | 126.9 | 129.9 | 552.3 | 525.4 |
| 60 | 60.5 | 378.7 | 673.5 | 187.2 | 183.6 | 654.0 | 873.4 |
| 75 | 76.7 | 379.9 | 776.2 | 249.9 | 226.3 | 726.9 | 1144.6 |
| 90 | 88.6 | 384.5 | 823.6 | 300.6 | 271.3 | 880.2 | 1429.8 |
| 105 | 101.0 | 386.9 | 888.1 | 373.1 | 302.6 | 1021.9 | 1569.7 |
| 120 | 111.7 | 391.7 | 920.4 | 425.3 | 338.4 | 1146.3 | 1730.3 |

TABLE X
The permeation of Lidocaine and Prilocaine from mucoadhesive
film (sample F8-F10) through sublingual mucosa ex vivo

| | PERMEATION ENHANCER TESTED | | | | | |
|---|---|---|---|---|---|---|
| | (μg/mL Lidocaine permeated) | | | (μg/mL Prilocaine permeated) | | |
| | without | | Transcutol | without | | Transcutol |
| Time | permeation | Transcutol | HP 10% + | permeation | Transcutol | HP 10% + |
| (min) | enhancers | HP 10% | Eugenol 6% | enhancers | HP 10% | Eugenol 6% |
| 0 | 0.1 | 0.0 | 9.2 | 0.0 | 0.0 | 0.0 |
| 15 | 15.9 | 203.5 | 165.1 | 30.7 | 71.7 | 55.4 |
| 30 | 32.4 | 320.1 | 256.8 | 59.3 | 133.5 | 104.9 |
| 45 | 41.7 | 392.6 | 381.2 | 114.7 | 192.2 | 268.6 |
| 60 | 58.7 | 431.0 | 394.4 | 141.4 | 214.0 | 314.5 |
| 75 | 71.2 | 446.3 | 440.4 | 155.3 | 240.8 | 392.2 |
| 90 | 83.7 | 490.5 | 466.2 | 173.3 | 283.3 | 450.0 |
| 105 | 95.7 | 477.0 | 495.7 | 201.8 | 270.0 | 514.8 |
| 120 | 100.5 | 493.4 | 514.8 | 231.4 | 281.4 | 557.2 |

### Example IV

### Pharmacokinetic Study of Lidocaine Hydrochloride Formulated in Mucoadhesive Film of the Invention

The pharmacokinetic profile of lidocaine hydrochloride formulated as a mucoadhesive film was determined using a pharmacokinetic model in piglets (Sus scrofa domesticus). Blood plasma concentrations of lidocaine hydrochloride were measured following administration of the film formulations to the gum in the area of front teeth (mandibula). The absorption of lidocaine into blood circulation confirms the local absorption into the deeper layers of the tissue.

### The Synergic Effect of Permeation Enhancers—Transcutol and Eugenol on Systemic Absorption oOf Lidocaine

The mucoadhesive film was formulated as a bi-layered film. The size of the film layer containing lidocaine hydrochloride used in the experiment was 1 cm×2 cm. One mucoadhesive film was applied (the composition of the film layer containing lidocaine hydrochloride was 10 mg of lidocaine hydrochloride with Transcutol® HP or Transcutol® HP in combination with eugenol as permeation enhancers).

The following formulations of lidocaine hydrochloride mucoadhesive films were tested:

Mucoadhesive layer was formulated by solvent casting method (polymer swelling/dissolution conditions: 2 hrs at 25° C.; solvent evaporation: 2 hrs at 60° C.) using ethanol and water (80/20% v/v) 0.331 ml/cm².

TABLE XIa
Composition of film containing Lidocaine HCl, Prilocaine HCl

| | % of dry | |
|---|---|---|
| Material | composition | mg/cm² film |
| Lidocaine Hydrochloride | 27.67 | 5.07 |
| Prilocaine Hydrochloride | 27.67 | 5.07 |
| Pharmacoat ® 606 | 26.40 | 4.84 |
| Klucel ™ EF | 13.20 | 2.42 |
| Eugenol | 0.00 | 0.00 |
| Transcutol ® HP | 0.00 | 0.00 |
| Glycerol | 5.06 | 0.93 |
| Total | 100.00 | 18.33 |

TABLE XIb
Composition of film containing Lidocaine HCl,
Prilocaine HCl and Transcutol ®

| | % of dry | |
|---|---|---|
| Material | composition | mg/cm² film |
| Lidocaine Hydrochloride | 24.76 | 5.07 |
| Prilocaine Hydrochloride | 24.76 | 5.07 |
| Pharmacoat ® 606 | 23.62 | 4.84 |
| Klucel ™ EF | 11.81 | 2.42 |
| Eugenol | 0.00 | 0.00 |
| Transcutol ® HP | 10.03 | 2.05 |
| Glycerol | 5.02 | 1.03 |
| Total | 100.00 | 20.48 |

TABLE XIc
Composition of film containing Lidocaine HCl, Prilocaine
HCl, Transcutol ® and Eugenol

| | % of dry | |
|---|---|---|
| Material | composition | mg/cm² film |
| Lidocaine Hydrochloride | 22.57 | 5.07 |
| Prilocaine Hydrochloride | 22.57 | 5.07 |
| Pharmacoat ® 606 | 21.53 | 4.84 |
| Klucel ™ EF | 10.77 | 2.42 |
| Eugenol | 7.37 | 1.66 |
| Transcutol ® HP | 10.03 | 2.25 |
| Glycerol | 5.16 | 1.16 |
| Total | 100.00 | 22.47 |

### Conclusion:

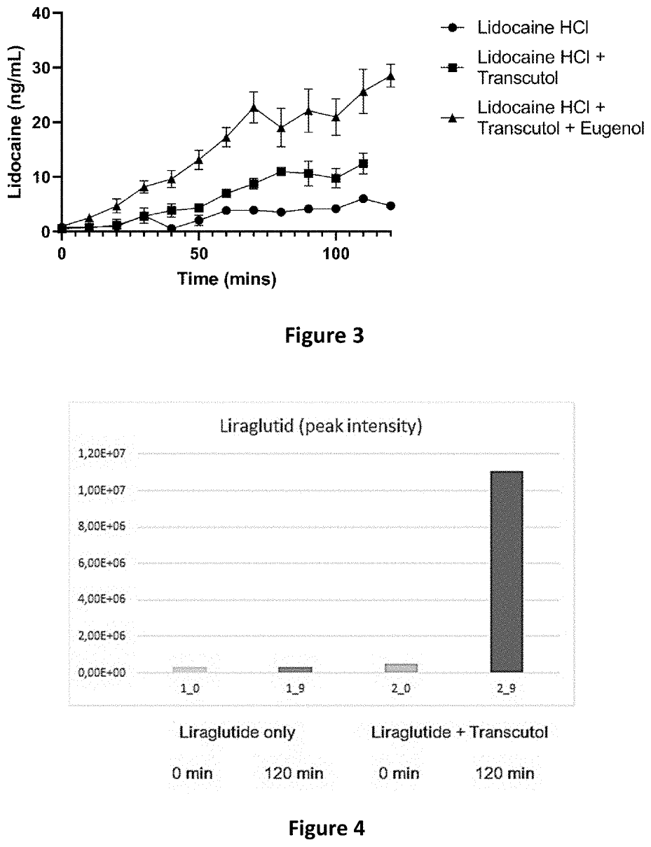

FIG. 3 shows pharmacokinetic profile of lidocaine following the administration using mucoadhesive film (the synergistic effect of Transcutol® HP and eugenol). The effective absorption of lidocaine through the oral mucosa of the gum was shown from the tested mucoadhesive film using a porcine animal model. Moreover, the effect of Transcutol® and Eugenol as permeation enhancers were clearly shown: from the data in FIG. 3. it can be concluded that absorption/permeation enhancer —combination of Transcutol® and eugenol—significantly promotes local absorption, and leads to increase in plasma concentration of lidocaine. This is also confirmed by Example III showing the effect of permeation enhancer ex vivo. Moreover, synergistic effect of enhanced permeation when combining Transcutol® and eugenol was confirmed by FIG. 3.

### Liraglutide

### Example V

### Ex Vivo Permeation of Liraglutide through Porcine Sublingual Mucosa

The objective of this study was to prove the effect of absorption enhancers of the invention on the permeation of solution of liraglutide through excised sublingual porcine mucosa ex vivo using a Franz diffusion cell model.

### Methods

Porcine sublingual mucosa was removed immediately after sacrifice. The mucosa was then prepared and mounted to the diffusion cell with the receptor compartment having a volume of 5 mL, containing phosphate buffer pH 8.5. The solution of liraglutide, and solution of liraglutide and permeation enhancer was tested. The concentration of liraglutide in the donor compartment corresponded to the 3 mg/mL of liraglutide.

The permeation was confirmed by measuring the presence of liraglutide in the receptor compartment under constant mixing at 37° C. Samples were taken at 0 and 120 minutes to determinate the presence of liraglutide in the receptor chamber. The concentration level of liraglutide was determined using HPLC/MS method.

The following solutions of lidocaine HCl were tested: liraglutide (3 mg/mL)+Transcutol® HP (10% solution).

### Results

FIG. 4 shows permeation of liraglutide through porcine sublingual mucosa ex vivo. The significantly increased permeation of liraglutide through sublingual mucosa ex vivo when combined with permeation enhancer, Transcutol® HP, when compared with liraglutide solution alone shows the unexpected efficacy of permeation enhancers of the invention.

1. An oromucosal film composition comprising:
a therapeutically effective amount of one or more active pharmacutical ingredients selected from the group consisting of fluticasone, liraglutide, glucagone, midazolam, diazepam, lidocaine, articaine, bupivacaine, prilocaine, mepivacaine, epinephrine and salts thereof;
a pharmaceutically acceptable polymer; and
an absorption enhancer selected from the group consisting of diethylene glycol monoethyl ether, eugenol, bile acids, caprylocaproyl polyoxyl-8 glycerides, disodium ethylenediaminetetraacetic acid, salt and mixtures thereof,
wherein the composition comprises at least diethylene glycol monoethyl and eugenol as absorption enhancers when it comprises epinephrine alone or together with one or more of lidocaine, articaine, bupivacaine, prilocaine, mepivacaine as the active pharmacutical ingredients.

2. The composition of claim 1 wherein the polymer is selected from the group consisting of hypromellose, hydroxypropyl cellulose, hydroxyethyl cellulose, carboxymethyl cellulose, carboxymethyl cellulose sodium, carbomer, polycarbophil, povidone, polyvinyl alcohol, polyethylene oxide, polyethylene glycol, sodium alginate, calcium alginate, xanthan gum, pectin, hyaluronic acid, sodium hyaluronate, tragacanth, guar gum, acacia gum, arabic gum, lectin, starch, gelatin, pullulan, carrageenan, chitosan, amino methacrylate copolymers, poloxamer, collagen, poly-amino acids, and mixtures thereof.

3. (canceled)

4. The composition of claim 1 wherein the composition comprises epinephrine as solid particles having a particle size in the range of from about 0.01 um to about 100 um.

5. The composition claim 1 wherein the weight ratio of the total active pharmacutical ingredient to polymer is from about 15% to about 250%.

6. (canceled)

7. The composition of claim 1 wherein the composition further comprises one or more pharmaceutically acceptable excipients selected from the group consisting of pH modifiers, disintegrants, plasticizers, saliva stimulating agent, taste masking agents, flavoring agents, sweeting agent, coloring agent, antioxidants, stabilizers, anti-foaming and/or defoaming components and mixtures thereof.

8. (canceled)

9. The composition of claim 1 wherein the composition is a bi-layered film where a first layer comprises one or more active pharmaceutical ingredients dispersed or dissolved in and/or disposed on a polymeric mucoadhesive layer, and a second layer which acts as a backing layer.

10. The composition of claim 1 wherein the composition is a tri-layered film comprising:
a layer comprising one or more active pharmaceutical ingredients dispersed or dissolved in and/or disposed on a polymeric layer;
a mucoadhesive layer; and
a backing layer,
wherein either the mucoadhesive layer or the polymeric layer comprising the active pharmaceutical ingredients is the middle layer.

11. (canceled)

12. (canceled)

13. (canceled)

14. The composition of claim 9 wherein one or more of the pharmaceutically acceptable excipients are exclusively or partly present in the mucoadhesive layer.

15. The composition of claim 10 wherein at least one of the active pharmaceutical ingredient is present in the mucoadhesive layer.

16. A method for preparation of the composition of claim 1 wherein the method comprises solvent casting, hot melt extrusion, electrospinning, 3-dimensional or flexographic printing, spraying, combination of solvent casting and spraying, electro spraying, solution blow spinning, electroblowing, centrifugal spinning, or a combination of electrospinning and electrospraying.

17. (canceled)

18. A method for preparation of the composition of claim 1 wherein the method comprises:
providing a suspension or solution of one or more active pharmaceutical ingredients in a solvent/dispersant;
simultaneously or separately providing a suspension or solution of the polymer in a solvent/dispersant;
combining the active ingredient suspension/solution and the polymer suspension/solution if prepared separately; and
removing the solvent/dispersant to obtain the polymeric layer.

19. The method of claim 18 wherein the solvent is selected from the group consisting of lower alkyl alcohols, acetone, methyl acetate, ethyl acetate, isopropyl acetate, acetonitrile, heptane, tetrahydrofuran, water, alkaline water, aqueous buffers, and mixtures thereof.

20. The method of claim 19 wherein the alcohol is selected from the group consisting of methanol, ethanol, isopropyl alcohol, 2-propanol, 1-propanol 1-butanol, t-butanol, and mixtures thereof.

21. (canceled)

22. (canceled)

23. A method of treating diabetes in a subject in need thereof comprising administering to the subject a composition according to claim 1 wherein one or more of liraglutide, glucagon and salts thereof are selected as the active pharmacutical ingredients.

24. A method of treating epilepsy in a subject in need thereof comprising administering to the subject a composition according to claim 1 wherein one or more of diazepam, midazolam and salts thereof are selected as the active pharmacutical ingredients.

25. A method of providing local anesthesia to a subject in need thereof comprising administering to the subject a composition according to claim 1 wherein one or more of lidocaine, articaine, bupivacaine, prilocaine, mepivacaine, and salts thereof are selected as the active pharmacutical ingredients.

26. A method of treating and/or preventing tooth pain or mouth ulcers in a subject in need thereof comprising administering to the subject a composition according to claims 1 wherein one or more of lidocaine, articaine, bupivacaine, prilocaine, mepivacaine, and salts thereof as the active pharmacutical ingredients.

27. The method of claim 26 wherein the composition further comprises fluticasone.

28. The method of claim 25 wherein the composition further comprises a low dose of epinephrine or a salt thereof.

29. A method of treating anaphylactic shock or cardiac arrest in a subject in need thereof comprising administering to the subject a composition according to claim 1 wherein epinephrine or salts thereof are selected as the active pharmaceutical ingredients.

30. (canceled)
